(19) United States
(12) Patent Application Publication
(10) Pub. No.: US 2007/0179810 A1
(43) Pub. Date: Aug. 2, 2007

(54) Controls for antimicrobial use and infection

(76) Inventor: Edward L. Goodman, Dallas, TX (US)

Correspondence Address: KRAMER & AMADO, P.C., 1725 DUKE STREET, SUITE 240, ALEXANDRIA, VA, 22314, US

(21) Appl. No.: 11/649,912

(22) Filed: Jan. 5, 2007

Related U.S. Application Data
(60) Provisional application No. 60/756,191, filed on Jan. 5, 2006.

(51) Int. Cl. G06Q 10/00; A61B 5/00

(52) U.S. Cl. 705/2; 600/300

(57) ABSTRACT

The present invention relates to protocols, systems, programs and methods for the integrated management of risk in a health care environment. The present invention relates to the management of antimicrobial therapy. The present invention relates to the management of infection and surgical site infections. The present invention further relates to the management of antimicrobial therapy, the management including (a) converting a patient from intravenous administration to oral administration of at least one antimicrobial; (b) limiting patient prophylaxis to a pre-operative, post-operative, or combination thereof length of time; (c) restricting at least one antimicrobial from administration to said patient; or (d) a combination of components (a), (b) and (c).

### CROSS-REFERENCE TO RELATED APPLICATION

This application claims priority to U.S. Provisional Application No. 60/756,191, the contents of which are incorporated herein by reference in their entirety.

### FIELD OF THE INVENTION

The present invention relates to the management of antimicrobial (antibiotic) therapy. The present invention also relates in general to the prevention and management of infection. The present invention relates to the management of surgical site infections.

### BACKGROUND OF THE INVENTION

Health care environments are now experiencing a crisis in the realm of risk management. For example, over the past few decades, health care practitioners have increasingly realized that the overuse and misuse of antibiotics leads to microbes with increased resistance to the same antibiotics. Health care environments have also realized a continued rise in the number of nosocomial infections as a result of surgical and other invasive procedures. At the same time, health care practitioners have become aware of a variety of strategies and procedures for managing such risks. However, for a variety of reasons including the cost of implementation, most health care environments (e.g., hospitals) have failed to implement effective and/or comprehensive systems of risk management. In addition to reducing the level of patient care and placing patients at risk, the absence of effective risk management places the health care environment at risk with respect to, inter alia, a loss of competitive advantage and increased patient litigation.

Health care environments worldwide are faced with the rapid emergence and spread of antibiotic resistant microorganisms. See McGowan et al., “Control of antimicrobial resistance in the health care system”, Infect. Dis. Clin. North. Am., 11:297-311 (1997); Schwartz et al., “Preventing the emergence of antimicrobial resistance: a call for action by clinicians, public health officials, and patients”, JAMA, 278:944-945 (1997). While an estimated 26-53% of hospitalized patients receive at least one antibiotic, antimicrobial therapy for these patients is often inappropriate or incorrectly administered. Not only is the wrong antibiotic often prescribed, but health care practitioners commit errors with respect to dosage, frequency, route and duration of antibiotic. Moreover, other than providing broad guidelines, in the health care environment, there is typically little effective communication between the microbiology laboratory, where the likely effectiveness of any antibiotic may be assessed, and the administrators of antimicrobial therapy. Such lapses in therapy and communication results in, inter alia, the emergence of antibiotic-resistance microorganisms, increased patient mortality and increased health care costs.

Experts agree that the consequences of inappropriate or overuse of antimicrobials (antibiotics) are different from those of inappropriate or overuse of other drugs and procedures. While the overuse of another type of drug or procedure may harm an individual patient, the overuse of antimicrobials and the associated development of resistant organisms can affect the patients of an entire population, for example the patient in the next bed or even patients that have yet to be admitted. In fact, within the complex problem of antimicrobial resistance, experts also agree that antimicrobial drug exposure exerts selective pressure favoring the emergence of resistance. See Schwartz et al., “Preventing the emergence of antimicrobial resistance: a call for action by clinicians, public health officials, and patients”, JAMA, 278:944-945 (1997). Therefore, antibiotic use, appropriate or not, often leads to microbial resistance.

Still, national surveys suggest that 22-65% of antibiotic use within hospitals is inappropriate. See Fraser et al., “Antibiotic optimization: an evaluation of patient safety and economic outcomes”, Arch Intern Med, 157(15):1689-1694 (1997); Pelletier, “Hospital usage of parenteral antimicrobial agents: a gradated utilization review and cost containment program”, Infect Control, 6:226-230 (1985); Marr et al., “Guidelines for improving the use of antimicrobial agents in hospitals: a statement by the Infectious Diseases Society of America”, J Infect Dis, 157(5):869-876 (1988); Dunagan et al., “Antimicrobial misuse in patients with positive blood cultures”, Am J Med, 87:253-259 (1989). The effects of inappropriate antibiotic prescribing on microbial resistance to antibiotics, adverse drug events, and healthcare costs are well known to health care practitioners. Antimicrobial drug resistance results in increased morbidity, mortality, and cost of healthcare because multi-drug resistant bacteria are much more difficult to treat. A pharmaco-economic study at Duke University Medical Center showed that treating a primary nosocomial bloodstream infection due to methicillin-resistant Staphylococcus aureus is about three times as costly as treating a similar infection due to methicillin-sensitive Staphylococcus aureus. See Abramson et al., “Nosocomial methicillin-resistant and methicillin-sensitive Staphylococcus aureus primary bacteremia: at what costs?”, Infect Control Hosp Epidemiol, 20:408-411(1999). A joint statement by the Society for Healthcare Epidemiology of America and the Infectious Diseases Society of America contends that “appropriate antimicrobial stewardship that includes optimal selection, dose, duration of treatment, as well as control of antibiotic use, will prevent or slow the emergence of resistance among organisms.” Shlaes et al., “Society for Healthcare Epidemiology of America and Infectious Diseases Society of America Joint Committee on the Prevention of Antimicrobial Resistance: guidelines for the prevention of antimicrobial resistance in hospitals”, Clin Infect Dis, 25:584-599 (1997). Moreover, several published reports describe implementation of antibiotic control programs in response to an outbreak of drug resistant organisms. See Patterson et al., “Association of antibiotic utilization measures and control of multiple-drug resistance in Klebsiella pneumoniae”, Infect Control Hosp Epidemiol, 21 :455-458 (2000); White et al., “Effects of requiring prior authorization for selected antimicrobials: expenditures, susceptibilities, and clinical outcomes”, Clin Infect Dis, 25:230-239 (1997); Smith, “Decreased antimicrobial resistance after changes in antibiotic use”, Pharmacotherapy, 19(8 pt 2):129S-132S (1999). But still, improvement in the management of risk associated with antimicrobial therapy is desperately needed.

Health care environments are also faced with the occurrence of an increasing number of nosocomial or hospital acquired infections. Surgical site infections represent one of the most frequently reported nosocomial infections. A variety of factors are believed to contribute to the rise of infection, including patient vulnerability related, procedure related and post operative care related factors. Indeed, nosocomial infections are the result of a complex interaction between vulnerable hosts, invasive and other life-saving procedures, as well as system failures leading to errors in patient care and overuse of antibiotics which select for resistant organisms. Moreover, since nosocomial infections are currently reportable to the public in several states and soon likely to be reported in all states, such infections can be a major source of embarrassment for a health care facility. More important, the increased occurrence of such infections raises the costs of health care, and may also result in increases in patient care complications and/or patient death. Simply put, as patient outcomes deteriorate, the risk of litigation-increases. Fortunately, many nosocomial infections are preventable. Given the above risks associated with nosocomial infections, improvement in the management of infection is clearly needed.

A variety of additional factors exacerbate the problems of risk management faced by health care environments. For example, factors such as terrorist attacks with biologic or chemical agents, an influenza pandemic, a tsunami, earthquake or a plane crash, can impact the operations of a health care environment, thereby increasing the risk to in-patients even more. Additionally, the annual influenza epidemic that health care practitioners have come to expect can result in major dislocations of care in health care environments, such as cancellations of surgery and turning away of patients due to overcrowding. In sum, a variety of external factors may work to increase the risk to patients within the health care environment.

Despite the serious problems faced by health care practitioners, a variety of barriers to the implementation of effective risk management programs continue to exist. Perhaps the most important barrier has been a lack of understanding by (and education of) health care practitioners as to the meaning and importance of risk management. This lack of understanding has only been exacerbated by the fact that there are relatively few infectious disease doctors available to actively champion the need for improved infection control. Turf battles within health care environments have also contributed to the problem. For example, in many health care environments, pathologists control both the microbiology lab and infection control. Yet, where an effective risk management program may require changes in the operating procedures of the lab and/or infection control, pathologists may perceive the changes as relinquishment of control and, as a result, resist any change. Finally, resistance from “Big Brother” has acted as a barrier to implementation. That is, as a result of their training and experience, physicians are resistant to outside interference with their practices. As examples, witness the resistance of many physicians to the electronic medical record as well as the inability to follow evidence based guidelines.

There exists, therefore, a need for effective risk management in the health care environment. There is a need for improvements in the management of antimicrobial therapy. There is also a need for improvements in infection control. Furthermore, there is a need for an integrated risk management protocols, programs and systems in the health care environment, as well as improved methods for their implementation.

### SUMMARY OF THE INVENTION

The present invention relates to protocols, systems, programs and methods for the integrated management of risk in a health care environment. The present invention relates to protocols, systems, programs and methods for the management of antimicrobial therapy, infection, surgical site infection, microbiology laboratories, and so on.

In sum, the present invention relates to a protocol for an integrated management of medical risk, said protocol comprising: establishing at least one first intervention, said first intervention in integration with at least one second intervention; wherein said protocol provides said integrated management of medical risk.

The present invention relates to a protocol for management of medical risk, said protocol comprising: establishing a first intervention for management of antimicrobial therapy, said first intervention comprising: (a) converting a patient from intravenous administration to oral administration of at least one antimicrobial; (b) limiting patient prophylaxis to a pre-operative, post-operative, or combination thereof length of time; (c) restricting at least one antimicrobial from administration to said patient; or (d) a combination of components (a), (b) and (c).

The present invention also relates to a method for integrated management of medical risk, said method comprising: establishing at least one first intervention, said first intervention in integration with at least one second intervention; wherein said method provides said integrated management of medical risk.

The present invention further relates to a method for management of medical risk, said method comprising: establishing a first intervention for management of antimicrobial therapy, said first intervention comprising: (a) converting a patient from intravenous administration to oral administration of at least one antimicrobial; (b) limiting patient prophylaxis to a pre-operative, post-operative, or combination thereof length of time; (c) restricting at least one antimicrobial from administration to said patient; or (d) a combination of components (a), (b) and (c); wherein said method provides said management of medical risk.

### BRIEF DESCRIPTION OF THE DRAWINGS

Additional advantages and features of the present invention will become apparent from the subsequent description and the appended claims, taken in conjunction with the accompanying drawings, wherein:

FIG. 1 illustrates the CAMP intervention.

FIG. 2 illustrates the integration of the CAMP intervention and an infection control intervention.

FIG. 3 illustrates the integration of the CAMP intervention, a microbiology lab intervention and an infection control intervention.

FIG. 4 illustrates the integration of the CAMP intervention with a microbiology lab intervention.

### DETAILED DESCRIPTION OF THE PREFERRED EMBODIMENTS

The present invention provides for protocols, systems, programs and methods for the management of risk in a health care environment. The protocols, systems, programs and methods include interventions for the management of risk associated with antimicrobial therapy. The protocols, systems, programs and methods include interventions for the management of the risk of infection and/or surgical site infections. The protocols, systems, programs and methods also include interventions for the management of a microbiology lab. In a preferred embodiment, the present invention includes a plurality of integrated interventions (interventions established and/or implemented in integration), so as to provide for an integrated risk management protocol, system, programs and/or method in a health care environment.

By the term “intervention”, it is meant the means by which an element or component (e.g., an element or component which has associated risk) of a health care environment is managed. It will be recognized that an element or component of a health care environment includes, but is not limited to, antimicrobial therapy, infection control and the microbiology laboratory.

By the phrase “in integration”, it is meant to exist and/or function in communication with or with feedback between at least one other intervention, element or component of a health care environment.

By the phrase “nosocomial infection”, it is meant any infection that develops after admission of the patient to the health care environment, including any infection that develops within 48 to 72 hours after admission and was not incubating at the time of admission.

By the phrase “surgical site infection”, it is meant any infection (e.g., physician diagnosed infection) involving a surgical wound or the deeper anatomic spaces beneath it, excluding stitch abscesses.

By the phrase “health care environment”, it is meant any environment in which health care may be provided. Those or ordinary skill in the art will recognize that the phrase “health care environment” may include a variety of environments including, but not limited to, clinics, hospitals, laboratories, triage facilities, disaster relief areas, long-term health care facilities such as nursing homes, assisted living, skilled nursing units and home health care, and so on. It will also be recognized that the health care environment may be made up of a plurality of separate but possibly related components, for example a health care system or network made up of numerous hospitals.

It will be recognized that the phrase “surveillance” includes, but is not limited to, the monitoring and/or observation of an element or component of a health care environment, for example the monitoring and observation of infections (e.g., rates of infection), antimicrobial use (e.g., trends in antibiotic use) and resistance (e.g., trends in resistance).

Furthermore, it will be recognized that the meaning of the term “management” (of risk, therapy, infection and so on) includes, but is not limited to, preventing, decreasing and/or providing a desired level (of risk, therapy, infection, and so on). Antimicrobial Therapy Intervention

According to a preferred embodiment of the invention, the present invention relates to protocols, systems, programs, and/or methods for the management of antimicrobial therapy and/or the risk(s) associated with antimicrobial therapy.

In this regard, the present invention may include at least one intervention for the management of antimicrobial therapy. Interventions for the management of antimicrobial therapy may include, but are not limited to, interventions for the management of microbial resistance. For example, interventions for the management of antimicrobial therapy include requiring consultation with specialists before certain antibiotics may be administered. Interventions may also include the use of computer programs for the management of antimicrobial therapy. For example, interventions for the management of antimicrobial therapy may include interactive computer programs that provide health care choices based on historical and laboratory data.

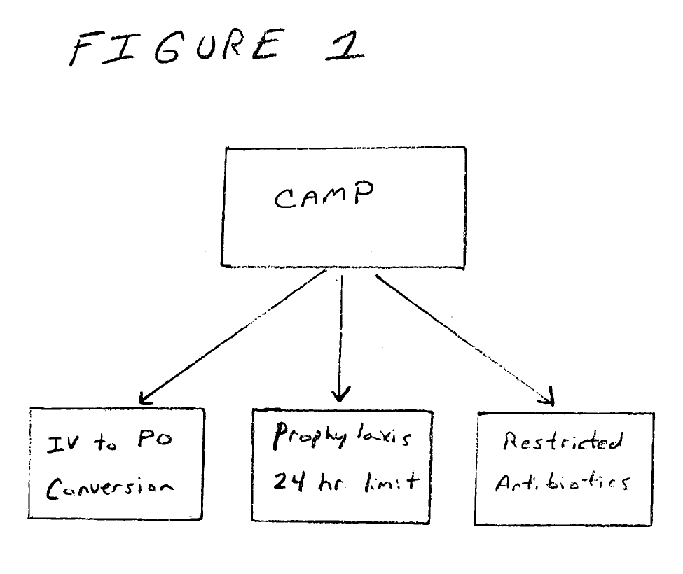

While the present invention is not limited to any particular intervention for the management of antimicrobial therapy, antimicrobial therapy and/or the risk(s) associated with antimicrobial therapy is preferably managed via an intervention referred to herein as the Comprehensive Antimicrobial Management Program (“CAMP”). As shown in FIG. 1, the main components of CAMP include, but are not limited to, (a) intravenous (IV) to oral (PO) conversion for highly bioavailable antimicrobials; (b) discontinuation of perioperative antimicrobial prophylaxis at 24 hours for clean and clean-contaminated procedures; and/or (c) restricted utilization of antibiotics which are high risk, high cost, have a high potential to promote resistance, or are “drugs of last resort.” More generally, the three main components of CAMP may include: (a) converting a patient from intravenous administration to oral administration of at least one antimicrobial; (b) limiting patient prophylaxis to a pre-operative, post-operative, or combination thereof length of time; (c) restricting at least one antimicrobial from administration to said patient; and/or (d) combinations thereof.

It will be recognized that the term “converting” of component (a) does not require that a patient is converted from IV to PO administration, but rather a patient may be converted from IV to PO administration, in view of criteria (e.g., the targeting of antimicrobials and/or screening of patients) as provided by the CAMP intervention, as discussed in more detail below.

It will also be recognized that the terms “discontinuation” or “limiting” of component (b) do not require that antimicrobial prophylaxis is discontinued or limited for a patient, but rather antimicrobial prophylaxis may be discontinued or limited for a patient, in view of criteria as provided by the CAMP intervention, as discussed in more detail below.

It will be further recognized that the term “restricting” of component (c) does not require any restriction with respect to use of any antimicrobial, but rather use of an antimicrobial may be restricted in view of criteria as provided by the CAMP intervention, as discussed in more detail below.

Therefore, in view of the discussion above and below, those of ordinary skill in the art will recognize that antimicrobial therapy and the risks associated therewith may be managed via the CAMP intervention and/or its components.

IV to PO Conversion—Certain antimicrobials are so well absorbed that they reach nearly equivalent blood levels whether they are given IV or PO. Thus, in accordance with the CAMP intervention, a variety of antimicrobials are preferably targeted for IV to PO conversion. Preferably, targeted drugs include those drugs that have an oral bioavailability such that serum levels approximate or equal those achieved from intravenous administration (e.g., clindamycin, fluconazole, levofloxacin, metronidazole, moxifloxacin and trimethoprimlsulfamethoxazole). It will be recognized that in the context of the present invention, the targeting of drugs may be done with or without consultation with and/or feedback from other elements of the health care environment, for example the Microbiology Laboratory or Infection Control.

In addition to targeting certain antimicrobials for IV to PO conversion, patients are preferably screened for IV to PO conversion. In one preferred embodiment of the present invention, the criteria for a patient to be converted from IV to PO administration include the patient having: (1) the ability to take oral medications or diet; (2) no persistent nausea, vomiting, or diarrhea; and/or (3) no disorder of the gastrointestinal tract that could decrease drug absorption. When these criteria are met, the patient is preferably converted from IV to PO administration, more preferably a clinical pharmacist orders this conversion. In certain preferred embodiments, after conversion, pharmacy records are monitored to determine if the conversion order is countermanded by a physician.

Preoperative Prophylaxis—Antimicrobial prophylaxis for certain types of surgery has been shown to decrease post-operative morbidity and infection rates. See Anon, “Antimicrobial prophylaxis in surgery”, Med Lett Drugs Ther, 43:92-97 (2001); American Society of Health-System Pharmacists, “ASHP Therapeutic Guidelines on Antimicrobial Prophylaxis in Surgery”, Am J Health Syst Pharm, 56:1839-1888 (1999); Mangram et al., “Guideline for prevention of surgical site infection, 1999. Hospital Infection Control Practices Advisory Committee”, Infect Control Hosp Epidemiol, 20:250-278 (1999). However, inappropriate and extended prophylaxis can lead to selection of resistant organisms. See Anon, “Antimicrobial prophylaxis in surgery”, Med Lett Drugs Ther, 43:92-97 (2001). In addition, the timing of prophylaxis may be important since adequate tissue concentrations of the antibiotic is preferably present at the time of incision and maintained until the incision is closed. See American Society of Health-System Pharmacists, “ASHP Therapeutic Guidelines on Antimicrobial Prophylaxis in Surgery”, Am J Health Syst Pharm, 56:1839-1888 (1999). Yet, this can usually be achieved with just one pre-operative dose. See Mangram et al., “Guideline for prevention of surgical site infection, 1999. Hospital Infection Control Practices Advisory Committee”, Infect Control Hosp Epidemiol, 20:250-278 (1999).

Accordingly, in one preferred embodiment of the CAMP intervention, patient prophylaxis is limited to a pre-operative, post-operative, or combination thereof length of time. Preferably, no more than 24 hours of prophylactic antimicrobials (antibiotics) are allowed. More preferably, if a patient undergoes a clean or clean-contaminated procedure, antimicrobial prophylaxis is discontinued after 24 hours. Preferably, the 24 hour period of allowed prophylaxis begins at the end of surgery. Those of ordinary skill in the art will understand that the phrase “clean surgery” implies incisions of the skin and subcutaneous tissue, whereas “clean contaminated surgery” implies transecting mucosal surfaces. Both “clean surgery” and “clean contaminated surgery” imply that no pre-existing infection is present. Preferably, pharmacy records are monitored for compliance with the discontinuation of antimicrobial prophylaxis.

Restriction of Antimicrobials—The CAMP intervention may also include restrictions on the administration of certain antimicrobials that are considered high risk, high cost, have increased potential to promote resistance, and/or are considered “drugs of last resort”. Those of ordinary skill in the art will recognize that the phrase “high risk” implies drugs with an inherent toxicity that precludes their use except in extreme or unusual circumstances where their risk is acceptable for the likely benefit achieved. The phrase “drugs of last resort” implies drugs that are broad spectrum, highly potent or impervious to common resistance mechanisms, and are used in treating highly drug resistant organisms or patients who are close to death from infection. Thus, those of ordinary skill in the art will recognize that a variety of antimicrobials may be subject to restriction (e.g., quinupristin/dalfopristin, imipenem, meropenem, voriconazole, echinocandins).

Accordingly, in one preferred embodiment of the present invention, certain antimicrobials are restricted from administration to a patient. Preferably, the administration of certain antimicrobials is discontinued after 48 hours of therapy. More preferably, after 48 hours, the administration of certain antimicrobials may only continue under the approval and/or guidance of an infectious disease physician. In this regard, for example, a pharmacist may remind the prescribing physician that after 48 hours, an infectious disease consult is required for continued administration of the antimicrobial. It is envisioned that the 48 hour period will allow the prescribing physician time to review culture and sensitivity results prior to deciding whether to discontinue the antimicrobial or consult an infectious disease physician. It is also envisioned that in certain circumstances, rather than consult an infectious disease physician, the prescribing physician may simply stop administration of the restricted antimicrobial and substitute administration of an antimicrobial that is not restricted. It will be recognized that a variety of antimicrobials may be subject to restriction including, but not limited to, those antimicrobials listed in Table 1 below.

In view of the above discussion, it will be recognized that in one preferred embodiment, the present invention is directed to a protocol for management of medical risk, said protocol comprising establishing a first intervention for management of antimicrobial therapy, said first intervention comprising: (a) converting a patient from intravenous administration to oral administration of at least one antimicrobial; (b) limiting patient prophylaxis to a pre-operative, post-operative, or combination thereof length of time; (c) restricting at least one antimicrobial from administration to said patient; or (d) a combination of components (a), (b) and (c). Preferably, the first intervention for management of antimicrobial therapy comprises components (a), (b) and (c), wherein all or some of the components may be established and/or implemented at any one time. Preferably, this protocol for the management of medical risk is included as part of a software program and/or a program and/or system of risk management.

In another preferred embodiment, the present invention is directed to a method for management of medical risk, said method comprising: establishing a first intervention for management of antimicrobial therapy, said first intervention comprising: (a) converting a patient from intravenous administration to oral administration of at least one antimicrobial; (b) limiting patient prophylaxis to a pre-operative, post-operative, or combination thereof length of time; (c) restricting at least one antimicrobial from administration to said patient; or (d) a combination of components (a), (b) and (c), wherein said method provides said management of medical risk. Preferably, the first intervention for management of antimicrobial therapy comprises components (a), (b) and (c), wherein all or some of the components may be established and/or implemented at any one time. Preferably, this method for the management of medical risk is included as part of a software program and/or a program and/or system of risk management.

In another preferred embodiment, the CAMP intervention (and components thereof) is in integration with other interventions and/or components of other interventions. Preferably, the CAMP intervention is established and/or implemented in integration with at least one second intervention. That is, the CAMP intervention preferably functions while in communication and/or receiving feedback from at least one other element or component of a health care environment, preferably at least one second intervention. For example, the CAMP intervention may be in integration with an intervention of the Microbiology Laboratory, wherein communication and/or feedback between the interventions allows health care practitioners to attribute antimicrobial resistance to the improper or overuse of a particular antimicrobial. The CAMP intervention (as well as the Microbiology Lab intervention) may also be in integration with an intervention of Infection Control, wherein the resulting feedback and/or communication between interventions may allow health care practitioners to identify the improper or overuse of an antimicrobial as a cause of an outbreak of infection within the health care environment. It should also be recognized that components of the CAMP intervention may be in integration with other interventions within the health care environment. For instance, in preferred embodiments, the restriction of antimicrobials component of CAMP may be in integration with an intervention of Infection Control, wherein certain antimicrobials may be administered only after consultation with an infectious disease physician. These concepts of in integration will be described in more detail below.

It is envisioned that the CAMP intervention will provide for significant advantages to health care environments including, but not limited to, reduced cost of health care, reduction in total antibiotic usage, shorter duration of hospital stay, reduced use of intravenous devices and complications thereof, as well as reductions in the frequency of antibiotic resistant organisms. It is envisioned that such advantages may be realized by the establishment and/or implementation of the CAMP intervention, for instance within risk management protocols, systems, programs, methods and so on. It is also envisioned that such advantages may be realized (even improved) by establishment and/or implementation of the CAMP intervention in integration with other interventions for the management of medical risk (risk associated with health care environments).

Infection Control Intervention

According to a preferred embodiment of the invention, the present invention relates to protocols, systems, programs and/or methods for managing infection (including the risk of infection).

In this regard, the present invention may include at least one intervention for managing infection. Interventions for the management of infection may include, but are not limited to, (a) standardizing definitions, polices and procedures; (b) targeted surveillance of surgical site infections; (c) targeted surveillance of intensive care unit infections; (d) rapid response to crises; (d) standardizing employee health procedures; and/or (e) combinations thereof. Such interventions are discussed in more detail below.

In a preferred embodiment, the intervention for the management of infection encompasses a standardization of definitions, polices and procedures, preferably within the health care environment. It will be understood that definitions, polices and procedures may be standardized within a single or a plurality of health care environments. For example, standardized policies and procedures are preferred for the reporting of infection rates, since comparisons between health care institutions or systems are inevitable. If two institutions define an infection differently, it is not possible to compare infection rates between the institutions. As a further example, the establishment of standardized systems for monitoring the adequacy of hand disinfection could reduce the risk of multiple nosocomial infections.

In a preferred embodiment, the intervention for the management of infection encompasses the targeted surveillance of surgical site infections. For example, it will be recognized that no hospital has the resources to monitor infection rates after all types of surgery. Accordingly, preferably only those surgeries that occur in large numbers or have higher risk of infection are targeted. When a health care system (e.g., a plurality of hospitals) wishes to target surgical sites, each member of the system (e.g., each hospital within the system) will preferably compare volume of procedures and rates of infections in order to select targets that are appropriate for the majority of institutions. It will be further recognized that targets may change from year to year depending on the results of targeted surveillance. For example, if cesarean sections are selected as a target, and after 12 months no infections are detected, a different target may be preferably selected for the next 12 month period. On the other hand, high risk procedures such as open heart surgery are preferably constantly surveyed.

In a preferred embodiment, the intervention for the management of infection encompasses the targeted surveillance of intensive care unit infections. For example, it will be recognized that intensive care unit infections are more dangerous because the patients are already quite ill to justify their presence in the intensive care unit (ICU). Moreover, since the variety of infections in ICU is so great given the wide variety of types of patients, preferably only central line associated blood stream infections (BSI) and ventilator pneumonia (VAP) are monitored or surveyed, at the recommendations of the CDC's National Healthcare Safety Network (NHSN). This surveillance is preferably accompanied by the accurate determination of all patients who have central lines or who are on ventilators. Such data may be collected daily. The infections may also be detected by predetermined, precise criteria. Rates are preferably reported over long enough time frames to establish meaningful statistics.

In a preferred embodiment, the intervention for the management of infection encompasses a rapid response to crises, preferably crises within the health care environment. Preferably, if a cluster of infections occur after a non-targeted surgical procedure, a policy exists for rapidly identifying the outbreak, determining the mechanism and instituting control measures. For example, outbreaks of post-operative infections commonly occur due to a particular microbial organism, but sometimes with greater frequency than is normal. With a rapid response mechanism in place, a rapid response team may be able to determine whether the infections are related to the surgery or to a member of the wound care team. If the infections are due to a member of the wound care team, that person may be identified and treated, and the outbreak halted.

In a preferred embodiment, the intervention for the management of infection encompasses the standardization of employee health procedures. It will be recognized that the health care of employees within the health care environment can impact a patient's health (and vice versa). For example, immunizations may protect employees from infections acquired from patients. Patients can also acquire infections from health care employees. Accordingly, immunization policies and procedures for health care employees are preferably planned and implemented with care, more preferably standardized within the heath care environment. Needle stick and body fluid exposures may also affect health care employees. Thus, standardized policies are preferably in place for the immunization of employees against Hepatitis B and for post-exposure prophylaxis to blood borne pathogens such as Hepatitis B and HIV. Moreover, standardized procedures of the detection of latent tuberculosis in employees may prevent transmission to patients, other employees and family members of employees.

In another preferred embodiment, the intervention for the management of infection (and/or components thereof) is in integration with other interventions and/or components of other interventions. Preferably, the intervention for the management of infection is established and/or implemented in integration with at least one second intervention. That is, the intervention preferably functions while in communication and/or receiving feedback from at least one other element or component of a health care environment, preferably at least one second intervention. The integration of the intervention for the management of infection control is described in detail throughout this application.

It is envisioned that the intervention for the management of infection will provide for significant advantages to health care environments including, but not limited to, reduced cost of health care, reduction in total antibiotic usage, shorter duration of hospital stay, reduced use of intravenous devices and complications thereof, as well as reductions in the frequency of antibiotic resistant organisms. It is envisioned that such advantages may be realized by the establishment and/or implementation of the intervention, for instance within risk management protocols, systems, programs, methods and so on. It is further envisioned that such advantages may be realized (even improved) by establishment and/or implementation of the intervention in integration with other interventions for the management of medical risk (risk associated with health care environments).

Surgical Site Infection Intervention

According to another preferred embodiment of the invention, the present invention relates to protocols, systems, programs and/or methods for managing surgical site infection (including the risk of surgical site infection).

In this regard, the present invention may include at least one intervention for managing surgical site infection. Interventions for managing surgical site infection may include, but are not limited to, non-pharmacologic interventions and/or pharmacologic interventions for managing surgical site infection. Non-pharmacologic interventions for managing surgical site infection may include, but are not limited to, (a) skin preparation prior to surgery; (b) supplemental oxygen during surgery; (c) maintenance of body temperature during surgery; (d) maintenance of blood sugar during and after surgery; and/or (e) combinations thereof. Pharmacologic interventions for managing surgical site infection may include, but are not limited to, (a) administration of perioperative antibiotics; (b) analysis of process procedures; (c) analysis of process outcomes; (d) modification of process based on the analysis of process procedures and outcomes; and/or (e) combinations thereof.

With respect to non-pharmacologic interventions, in a preferred embodiment, the intervention for managing surgical site infection encompasses skin preparation prior to surgery. It will be recognized that skin preparation prior to surgery includes, but is not limited to, proper hair removal and disinfection. Improper hair removal includes shaving with razors and hair removal the night before surgery. In contrast, clipping hair just prior to surgery is optimally preferred. Also, a variety of skin disinfectants are on the market, all of which vary in potency, length of protection and tolerability. Purchasing cooperatives among hospitals may favor one disinfectant product over another. Thus, Infection Control will preferably select the appropriate disinfectant and instruct staff on its proper use.

In a preferred embodiment, the intervention for managing surgical site infection encompasses supplemental oxygen during surgery. It will be recognized that supplemental oxygen during surgery may be effective in reducing post-operative infections. At least one study has shown that raising the inspired oxygen concentration reduces infection rate. See Grief et al., “Supplemental perioperative oxygen to reduce the incidence of surgical wound infections, N Eng J Med, 342:161-167 (2000).

In a preferred embodiment, the intervention for managing surgical site infection encompasses the maintenance of body temperature during surgery. It will be recognized that anesthesia contributes to the lowering of body temperature, whereas hypothermia contributes to higher infection rates, particularly in colon-rectal surgery. Accordingly, the preferred maintenance of normothermia may reduce post-operative infection rate.

In a preferred embodiment, the intervention for managing surgical site infection encompasses the maintenance of blood sugar during and/or after surgery. Preferably, during surgery and in ICU, blood sugar is controlled within certain limits, in an effort to improve outcomes including reduction of infection rates. See Furnary et al., “Continuous intravenous insulin infusion reduces the risk of wound infection in diabetics after open heart operations, Ann Thor Surg, 67:352-60 (1999).

With respect to pharmacologic interventions, in a preferred embodiment, the intervention for managing surgical site infection encompasses the administration of perioperative antibiotics. For example, it will be recognized that for selected clean surgical procedures, perioperative antibiotics are preferably administered, which may reduce the rate of post-operative infections. For the insertion of prosthetic devices, open heart surgery and surgery on the GI and female genital tract, antibiotics are also administered. Timing of administration may be critical since the optimal level of drug should be in the tissues at the time of incision. Prolongation of post-operative antibiotics is not only unhelpful, but it may increase the risk of selecting resistant organisms. See Bratzler et al., “Antimicrobial prophylaxis for surgery, Clin Infect Dis, 38:1706-15 (2004); Ibid., “The surgical infection prevention and surgical care improvement projects, CID, 43:322-330 (2006).

In a preferred embodiment, the intervention for managing surgical site infection encompasses the analysis of process procedures. For example, intraoperative processes are preferably monitored for compliance with measures that may reduce the rate of infection. Electronic monitors may be used to document hypoxemia, hypoglycemia and hypothermia. The collection of such data may be expedited by computerized electronic health care records. Preferably, variations in compliance are demonstrated and used in enhancing compliance. Preferably, processes may be modified based on the analysis of the process procedures.

In a preferred embodiment, the intervention for managing surgical site infection encompasses the analysis of process outcomes. For example, infection rates following surgical procedures are commonly and preferably monitored. Moreover, in many states, this information is published for public review. Variation in outcomes may be traced back to various causes, some of which may be monitored, for instance perioperative antibiotics and intraoperative monitoring for hypothermia, hyperglycemia and hypoxemia. Preferably, if clusters of infections are detected, searches for epidemic strains are performed along with process monitoring. Where preferable, processes may be modified based on the analysis of the process outcomes.

In another preferred embodiment, the intervention for managing surgical site infection encompasses the modification of process based on the analysis of process procedures and outcomes. Such modification is discussed above in more detail.

In another preferred embodiment, the intervention for managing surgical site infection (and/or components thereof) is in integration with other interventions and/or components of other interventions. Preferably, the intervention for managing surgical site infection is established and/or implemented in integration with at least one second intervention. That is, the intervention preferably functions while in communication and/or receiving feedback from at least one other element or component of a health care environment, preferably at least one second intervention. The integration of the intervention for managing surgical site infection is described in detail throughout this application.

It is envisioned that the intervention for managing surgical site infection will provide for significant advantages to health care environments including, but not limited to, reduction in total antibiotic usage, shorter duration of hospital stay, less use of intravenous devices and complications thereof, as well as reductions in the frequency of antibiotic resistant organisms. It is envisioned that such advantages may be realized by the establishment and/or implementation of the intervention, for instance within risk management protocols, systems, programs, methods and so on. It is further envisioned that such advantages may be realized (even improved) by establishment and/or implementation of the intervention in integration with other interventions for the management of medical risk (risk associated with health care environments). Laboratory Intervention

According to another preferred embodiment of the invention, the present invention relates to protocols, systems, programs and/or methods for management of a microbiology laboratory.

In this regard, the present invention may include at least one intervention for the management of a microbiology laboratory. Interventions for managing a microbiology laboratory may include, but are not limited to (a) standardizing laboratory procedures; (b) restricting cultures submitted to the laboratory; (c) restricting susceptibility reporting; and/or (e) combinations thereof.

In a preferred embodiment, the intervention for managing a microbiology laboratory encompasses the standardization of laboratory procedures. It will be recognized that the optimal care of patients requires adherence to certain laboratory standards. Unlike routine hematology and chemistry laboratories where automated procedures are similar throughout all hospitals, microbiology laboratories have more latitude and less automation. Specimens are more varied than just blood and urine. As a result, there is more variation among microbiology laboratories. However, with the requirement that infection rates are available to the public, microbiology laboratories preferably have standardized policies and procedures since microbiology reports directly affect antibiotic choices. Moreover, within a health care environment or system compromised of multiple hospitals, standardization is more preferred because of inter-hospital transfers and comparisons of infection rates.

In a preferred embodiment, the intervention for managing a microbiology laboratory encompasses restrictions on cultures submitted to the laboratory. It will be recognized that the variety of specimens submitted to microbiology laboratories leads to a preference for establishing guidelines for the proper collection and submission of specimens. For example, improperly collected voided urine from females can be contaminated with vaginal secretions. The resulting present organisms may be construed as a cause of disease when, in fact, they are normal bacteria. Also, the laboratory preferably includes policies on how to process specimens and report results. Otherwise, the laboratory may encourage the overuse and/or misuse of antibiotics. Similarly, the submission of respiratory secretions which are contaminated by colonizing organisms can lead to reports that encourage the misuse of antibiotics. Disclaimers in reporting is only one example of how a laboratory may discourage the overuse of antibiotics. As a further example, limitations on the reporting of contaminated blood cultures may reduce the administration of unnecessary and potentially dangerous antimicrobials.

In a preferred embodiment, the intervention for managing a microbiology laboratory encompasses restrictions on susceptibility reporting. It will be recognized that laboratories often report on the susceptibility of drugs even when those drugs are not effective in certain body locations. For example: (1) nitrofurantoin is an effective drug for many urinary tract infections, but ineffective in treating systemic infections. If a laboratory reports susceptibility to nitrofurantoin in a blood stream infection and, as a result, a physician uses that drug for a blood stream infection, the patient is receiving ineffective therapy; (2) bacteria that cause meningitis may be susceptible to drugs in the laboratory that do not penetrate into spinal fluid. Thus, if the laboratory reports these drugs as effective, physicians may select the drug for treatment, even though the drug does not reach the site of infection. Such a situation could easily lead to fatality; and (3) in the event of an outbreak of infection due to the overuse of an antibiotic, continued reporting of that drug in susceptibility tests may contribute to its continued use, when discontinued use is preferred. Accordingly, in view of at least the above, it is recognized that restrictions on susceptibility reporting may improve patient care.

In another preferred embodiment, the intervention for the management of a microbiology laboratory (and/or components thereof) is in integration with other interventions and/or components of other interventions. Preferably, the intervention for the management of a microbiology laboratory is established and/or implemented in integration with at least one second intervention. That is, the intervention preferably functions while in communication and/or receiving feedback from at least one other element or component of a health care environment, preferably at least one second intervention. The integration of the intervention for the management of a microbiology laboratory is described in detail throughout this application.

It is envisioned that the intervention for the management of a microbiology laboratory will provide for significant advantages to health care environments including, but not limited to, reduction in health care costs, reduction in total antibiotic usage, shorter duration of hospital stay, reduced use of intravenous devices and complications thereof, as well as reductions in the frequency of antibiotic resistant organisms. It is envisioned that such advantages may be realized by the establishment and/or implementation of the intervention, for instance within risk management protocols, systems, programs, methods and so on. It is further envisioned that such advantages may be realized (even improved) by establishment and/or implementation of the intervention in integration with other interventions for the management of medical risk (risk associated with health care environments).

Additional Intervention

It will be recognized that the present invention may encompass a variety of additional interventions for the improvement of patient health care. For example, without limitation, the present invention may encompass Computerized Physician Order Entry (CPOE), which has been mandated by regulatory agencies and will become the norm in the near future. Also, the Electronic Health Record (HER), which is the basis for CPOE, has the potential to improve integration of microbiology labs, infection control, antibiotic management and other interventions. In addition, a series of algorithms may be constructed to discourage physicians from ordering unnecessary cultures (e.g., urine cultures from non-symptomatic patients, tracheal cultures from patient who have fever but no evidence of respiratory infection). For instance, ordering an antibiotic could initiate a series of dialog boxes that would cite evidence based guidelines. In such a way, the overuse of antibiotics could be diminished. Also, additional interventions may include specialized protocols, systems, programs and/or methods for the management of terrorist attacks or natural disasters as well as protocols, systems, programs and/or methods for the management of influenza (and other) pandemics or epidemics.

Intervention Integration

The present invention also relates to integrated protocols, systems, programs and/or methods for the management of risk in a health care environment. The present invention may include at least one intervention (or a plurality of interventions) in integration, thereby providing for an integrated risk management protocol, system, program and/or method in a health care environment.

In preferred embodiments, the present invention includes at least one intervention in integration (e.g., established and/or implemented in integration) with at least one second intervention. Preferably, the present invention includes a plurality of interventions, wherein the plurality of interventions in integration provides for an integrated management of risk.

In accordance with the inventions described herein, it will be recognized that, in a health care environment, the successful development of health care programs may be best realized by interactions between a plurality of elements within the environment. For example, it has now been recognized that the development of programs for the control of infection and antibiotic use is most successful when there is interaction (e.g., communication and/or feedback) between Infection Control, Antimicrobial Programs and the Microbiology Laboratory. Preferably, the persons responsible for developing clinically relevant Microbiology Laboratory procedures interact with Infection Control and Antimicrobial Programs. It is towards examples such as this that the “in integration” aspect of the present invention is preferably directed.

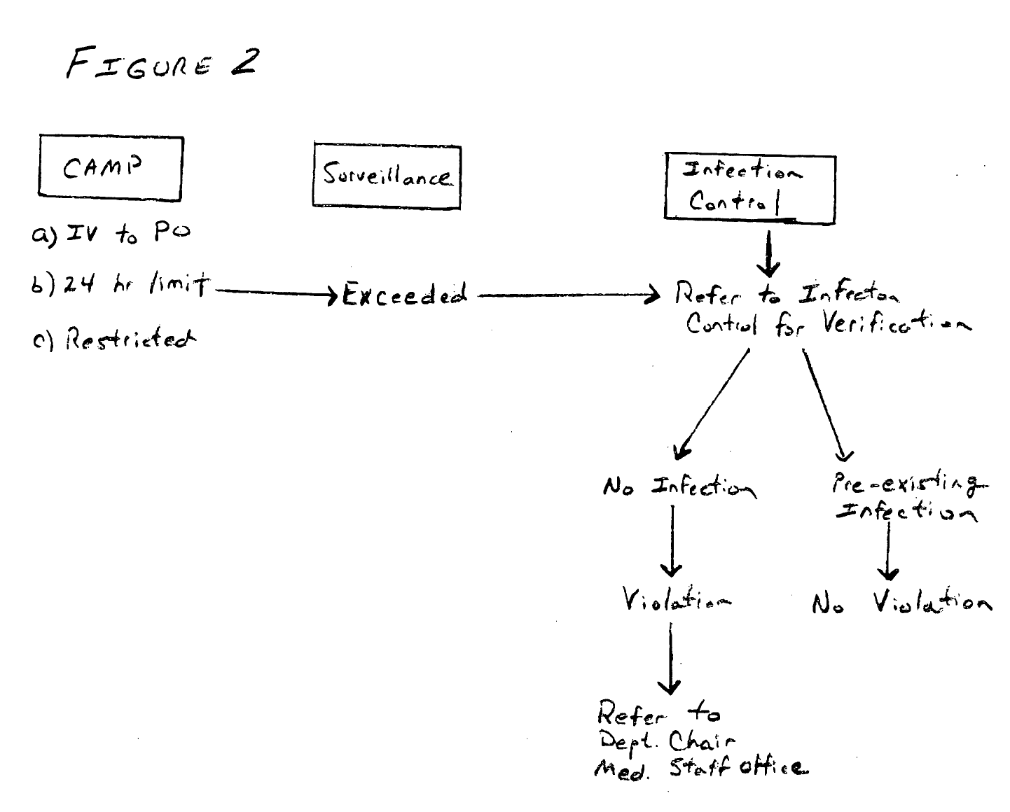

As illustrated in FIGS. 2-4, the present invention preferably encompasses the integration of the CAMP intervention with an Infection Control intervention and/or a Microbiology Lab intervention.

In one preferred embodiment, as illustrated in FIG. 2, the CAMP intervention is integrated with an Infection Control intervention. As discussed above, one component of the CAMP intervention is the discontinuation of prophylactic antimicrobials after 24 hours for clean surgery. As illustrated in FIG. 2, if surveillance detects a physician is prolonging prophylaxis beyond 24 hours, the CAMP intervention sends the patient data to an Infection Control Microbiologist. Upon receipt of patient data, in consultation with CAMP, the Infection Control Microbiologist reviews all operation notes, pathology reports and any operation cultures, in order to determine if a preexisting infection existed. After this review, if pre-operation infection is suspected, the Infection Control Microbiologist may determine that there has been no violation of the CAMP intervention. However, if there is no evidence of an infection being treated, the Infection Control Microbiologist may determine that a violation of the CAMP intervention has occurred. The violation of the CAMP intervention may then be reported to appropriate supervising authorities (e.g., Department Chair and/or Medical Staff Office). Through such integration, the management of antimicrobial therapy and infection control may be strengthened and, as a result, the health care environment improved with respect to patient care.

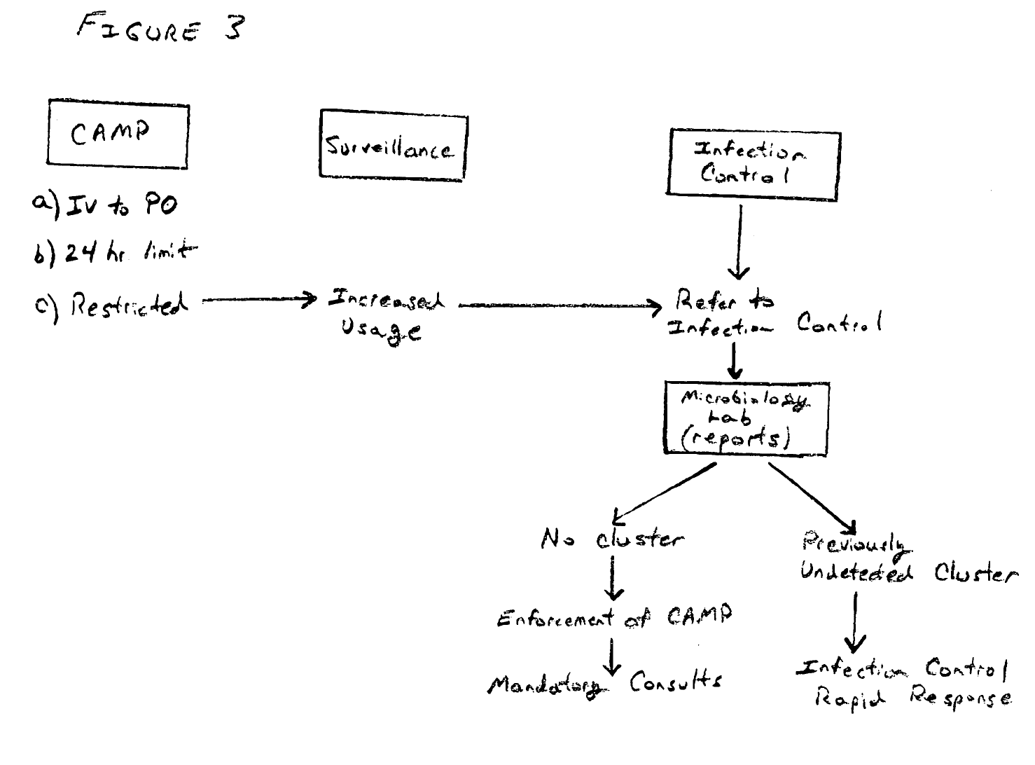

In another preferred embodiment, as illustrated in FIG. 3, the CAMP intervention is integrated with Infection Control and Microbiology Laboratory interventions. As discussed above, one component of the CAMP intervention is restricted use of certain antimicrobials. As illustrated in FIG. 3, if surveillance detects an increased usage of a particular antimicrobial, the increased usage is referred to Infection Control. In consultation with CAMP, Infection Control then reviews all reports from the Microbiology Laboratory to determine whether the increase usage is due to a certain unit of the health care environment (e.g., nursing unit) or an unsuspected outbreak or epidemic (cluster). If it is determined that the increased usage is due to a previously undetected outbreak or epidemic (cluster), a rapid Infection Control response may be instituted. However, if it is determined that the increased usage is due to a certain unit of the health care environment, Infection Control may require more stringent enforcement of CAMP intervention components. Through such integration, the management of antimicrobial therapy and infection control may be strengthened and, as a result, the health care environment improved with respect to patient care.

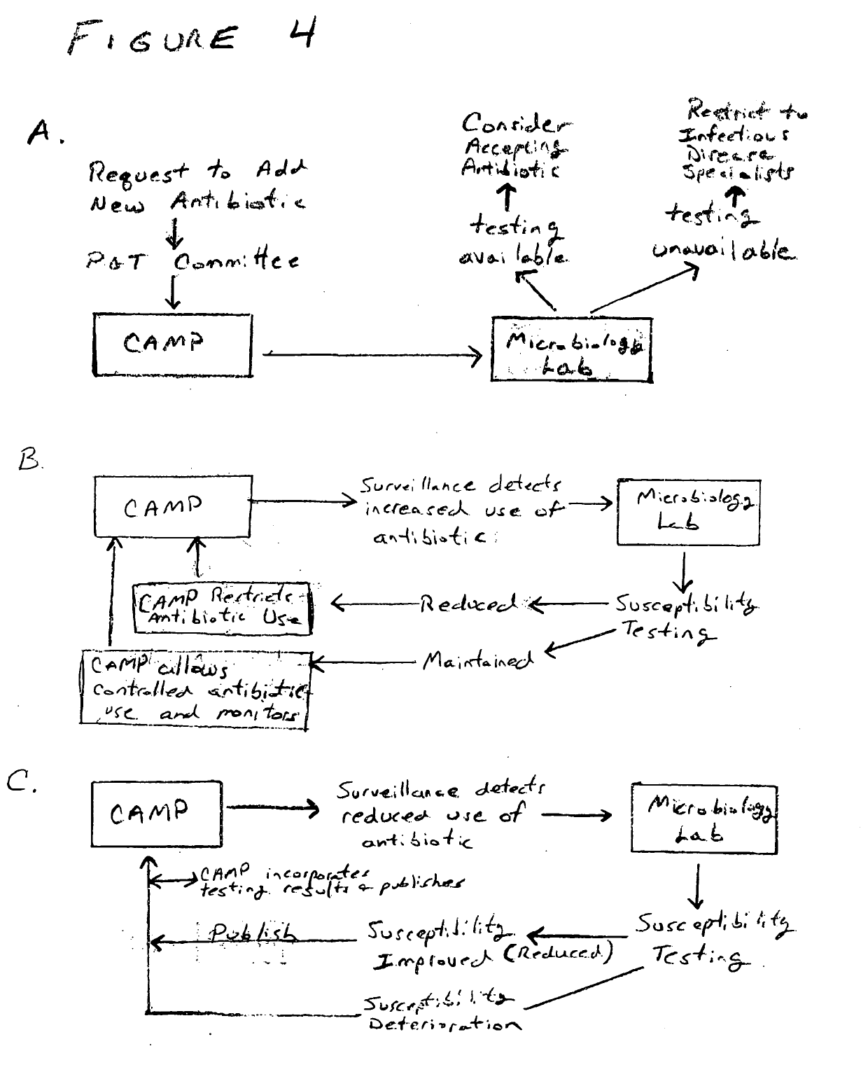

In further preferred embodiments, as illustrated in FIG. 4, the CAMP intervention is integrated with Microbiology Laboratory interventions. In a first example, as illustrated in FIG. 4A, the CAMP intervention may wish to allow a new antimicrobial on formulary (add a new antibiotic). However, in consultation with CAMP, the Microbiology Laboratory must have the ability to perform susceptibility tests on bacteria against this new antimicrobial. If the Microbiology Laboratory is unable to perform such testing, the lab will restrict use of the antimicrobial to Infectious Disease specialists only. It such testing is available, the lab may allow use of the antimicrobial by non-Infectious Disease specialists. In a second example, as illustrated in FIG. 4B, the CAMP intervention wishes to determine if use of an antimicrobial has had any effect on resistance to that antimicrobial. In consultation with CAMP, the Microbiology Laboratory compares susceptibility reports and correlates changes with increased or decreased use of the antimicrobial within CAMP. Through such integration, the management of antimicrobial therapy and the microbiology lab may be strengthened and, as a result, the health care environment improved with respect to patient care.

In view of the above discussion, it will be recognized that in one preferred embodiment, the present invention encompasses protocols, systems, programs and/or methods for an integrated management of medical risk (and/or health care environment), comprising: establishing at least one first intervention, said first intervention in integration with at least one second intervention; wherein said protocol provides said integrated management of medical risk. Preferably, the first intervention is established and/or implemented in integration with the second intervention.

Preferably, the protocols, systems, programs and/or methods for an integrated management include a plurality of interventions, wherein said plurality of interventions in integration provides for said integrated management of medical risk. Preferably, the plurality of interventions are established and/or implemented in integration.

Preferably, the protocols, systems, programs and/or methods for an integrated management of are included as part of a software program or a system of risk management.

Preferably, the first or second intervention is an intervention for management of antimicrobial therapy. More preferably, the first or second intervention is an intervention for management of antimicrobial therapy which includes at least one of the following: (a) converting a patient from intravenous administration to oral administration of at least one antimicrobial; (b) limiting patient prophylaxis to a pre-operative, post-operative, or combination thereof length of time; (c) restricting at least one antimicrobial from administration to said patient; and (d) combinations thereof.

Preferably, the first or second intervention is an intervention for infection control. More preferably, the first or second intervention is an intervention for infection control which includes at least one of the following: (a) standardizing definitions, polices and procedures; (b) targeted surveillance of surgical site infections; (c) targeted surveillance of intensive care unit infections; (d) rapid response to crises; (d) standardizing employee health procedures; and (e) combinations thereof.

Preferably, the first or second intervention is an intervention for control of surgical site infection. The intervention for control of surgical site infection may be a non-pharmacologic intervention, preferably a non-pharmacologic intervention which includes at least one of the following: (a) skin preparation prior to surgery; (b) supplemental oxygen during surgery; (c) maintenance of body temperature during surgery; (d) maintenance of blood sugar during and after surgery; and (e) combinations thereof. The intervention for control of surgical site infection may be a pharmacologic intervention, preferably a pharmacologic intervention which includes at least one of the following: (a) administration of perioperative antibiotics; (b) analysis of process procedures; (c) analysis of process outcomes; (d) modification of process based on the analysis of process procedures and outcomes; and (e) combinations thereof.

Preferably, the first or second intervention is an intervention for laboratory control. More preferably, the first or second intervention is an intervention for laboratory control which includes at least one of the following: (a) standardizing laboratory procedures; (b) restricting cultures submitted to the laboratory; (c) restricting susceptibility reporting; and (e) combinations thereof.

In another preferred embodiment, the present invention encompasses protocols, systems, programs and/or methods for an integrated management of medical risk (and/or health care environment), comprising: establishing a first intervention for management of antimicrobial therapy, said first intervention comprising: (a) converting a patient from intravenous administration to oral administration of at least one antimicrobial; (b) limiting patient prophylaxis to a pre-operative, post-operative, or combination thereof length of time; (c) restricting at least one antimicrobial from administration to said patient; or (d) a combination of components (a), (b) and (c), wherein said first intervention is implemented in integration with at least one second intervention. Preferably, the protocols, systems, programs and/or methods is included as part of a software program or a system of risk management.

It is envisioned that the integrated interventions of the present invention are able to provide for signification advantages in a health care environment. It is envisioned in particular that the advantages may be realized through the integration of various aspects (programs) of the health care environment, whether through integrated protocols, systems, programs, methods (and so on) of risk management. For example, it is envisioned that integration can result in reductions in health care cost, reductions in total antibiotic usage, shorter duration of hospital stay, reduced use of intravenous devices and complications thereof, as well as reductions in the frequency of antibiotic resistant organisms. It is emphasized that not only are the personnel involved with integrated programs able to share observations and data with other involved persons, but rapid interaction and feedback between components of an integrated approach allows for a more timely resolution of problems. Shared responsibility leads to improved outcomes, and stakeholders enhance the efficiency of the systems.

Those of ordinary skill in the art will recognize that the methodology described herein (e.g., interventions and integration) may be included or incorporated within a variety of protocols, systems, programs and methods, preferably for the management of risk in a health care environment. Those of ordinary skill in the art will also recognize that the methodology described herein may be included or incorporated within software programs and/or systems of risk management, preferably in a health care environment.

### EXAMPLES

Additional advantages and features of the present invention will become apparent from the subsequent examples, taken in conjunction with the prior description and the appended claims, wherein:

A study was designed and conducted to test the hypothesis that in a hospital, antimicrobial use could be improved via the present inventions described herein. The improvements were envisioned to include reductions in overall antibiotic use, cost savings and decreased antimicrobial resistance.

Design: interventional study with historical cohort. Three categories of inpatient antibiotic orders were monitored beginning April 2001. Data were analyzed after the first 33 months. Patient outcomes were reviewed during the hospital stay and at readmission if readmission occurred within 30 days.

Setting: a general community, not-for-profit, 900 bed hospital with residents in medicine, surgery, obstetrics-gynecology, and psychiatry.

Participants: physicians who ordered any of the targeted antibiotics.

Interventions: The Antimicrobial Therapy intervention included CAMP: conversion from intravenous to oral administration for selected highly bioavailable antibiotics; perioperative prophylaxis discontinued at 24 hours for clean surgery; and requesting an infectious disease physician consultation for selected drugs.

The Infection Control intervention included the detection of pre-operative infection that justified continued use of post-operative antibiotics. Alternatively, an absence of pre-operative infection encouraged the discontinuation of antibiotics after the 24 hour period.

The Microbiology Lab intervention included the monitoring of antimicrobial resistance patterns as influenced by reductions in antibiotic use.

Results: from April 2001 through December 2003, 1426 antimicrobial orders meet the criteria for intervention implementation. Overall physician compliance with the program was 76%, ranging from 57% for perioperative prophylaxis to 92% for IV to oral conversion. Antimicrobial cost per patient census day decreased by 31 % from $13.67 in 2000 (prior to program implementation) to $9.41 in 2003. Total acquisition cost savings were $1,841,203 for the three year period. In addition, resistance of Klebsiellu pneumoniae to numerous antibiotics was significantly reduced.

Conclusion: the management of risk (e.g., of antimicrobial therapy) in a hospital was successful.

Methods

In April 2001, a multidisciplinary Antimicrobial Management Team was formed. The team was comprised of the Chief of Infectious Diseases, two clinical pharmacists, and a microbiologist. The goals of the team were to improve antibiotic usage, prevent and for slow the emergence of resistant organisms, improve patient outcomes, and decrease healthcare costs. The design was an interventional study using historical comparison. The Comprehensive Antimicrobial Management Program (CAMP) was approved by the Pharmacy and Therapeutic Committee and the Medical Board of the hospital. The medical staff was notified about the Program at section or department meetings, teaching conferences, and through the Pharmacy Newsletter.

The three components of CAMP included: (1) intravenous (IV) to oral (PO) conversion for highly bioavailable antimicrobials, (2) discontinuation of perioperative antimicrobial prophylaxis at 24 hours for clean and clean-contaminated procedures, and (3) restricted utilization of antibiotics which are high risk, high cost, have a high potential to promote resistance, or are “drugs of last resort.” In order to evaluate compliance with the above components, a report using the pharmacy computer system was generated daily to identify patients receiving the targeted antimicrobials.

IV to PO Conversion

The following antimicrobials were targeted for IV to PO conversion: clindamycin, fluconazole, levofloxacin, metronidazole, moxifloxacin and trimethoprimlsulfamethoxazole. The criteria for a patient to be switched from IV to PO antimicrobials were: (1) ability to take oral medications or diet, (2) no persistent nausea, vomiting, or diarrhea, and (3) no disorder of the gastrointestinal tract that could decrease drug absorption. When these three criteria were met, a clinical pharmacist wrote an order on the patient's chart changing the antibiotic from the IV to PO route. A note was placed on the patient's chart notifying the physician of the change. Pharmacy records were followed to determine if the order to convert to PO was countermanded by the physician.

Preoperative Prophylaxis

The study allowed up to 24 hours of prophylactic antibiotics. If the patient underwent a clean or clean-contaminated procedure, a note was left on the chart asking the physician to discontinue antimicrobial prophylaxis after 24 hours. Compliance with this request was monitored by review of pharmacy records.

Restriction of Antimicrobials

Under the study, quinupristin/dalfopristin could only be prescribed by an infectious disease physician. Seven other antimicrobial agents (listed in Table 1) were restricted to infectious disease physicians after 48 hours of therapy. A pharmacist left a note on the patient's chart reminding the prescribing physician that after 48 hours, an Infectious Disease consult was required for continuation of the antimicrobial. The 48 hour period allowed the physician to review culture and sensitivity results prior to deciding whether to discontinue the antibiotic or consult an infectious disease physician. It was assumed that infectious disease physicians would be more judicious in the use of these drugs but no formal strategy was employed to ensure this. In most cases, the prescribing physician stopped the drug and substituted another that was not restricted. In only a minority of cases, was an infectious disease physician consulted.

### Controlling Antimicrobial Use in a Community Hospital

TABLE 1
ANTIMICROBIALS RESTRICTED TO INFECTIOUS DISEASE
PHYS: CLANS AFTER 48 HOURS

| | |
|---|---|
| Vancomycin | Linezolid (Zyvox ®) |
| Cefepime (Maxipime ®) | Ceftazidime |
| Imipenem (Primaxin ®) | Meropenem (Merrem ®) |
| Ertapenem (Invanz ®) | |

Data Collection

Data on annual antimicrobial doses charged, annual antimicrobial acquisition costs, and patient census days were obtained from the Finance Department. Antimicrobial doses in grams were converted to Daily Defined Doses (DDD) by using published conversion factors. See NNIS System, National Nosocomial Infections Surveillance (NNIS) System Report, data summary from January 1992 through June 2003, issued August 2003, Am J Infect Control, 3 1(8):481-498 (2003). An analysis of variance (ANOVA) was performed comparing the mean DDD per 1000 patient days between the years 2000 (prior to program implementation), 2001 (first year of CAMP), 2002 (second year of CAMP) and 2003 (third year of CAMP). A value of p<0.05 was considered significant. 2001 drug costs were used uniformly for all cost calculations so that inflation or deflation would not be a factor.

The Microbiology Department collected data on the susceptibility of major pathogens and published an annual antibiograrn, a copy of which was placed in each hospital chart as well as sent to all physicians. Data from the year prior to implementing the program (2000) was compared to data collected during the second year (2002) and third year (2003) of the program. T tests were used in determining statistical significance between the years.

The Results

Table 2 lists the number of interventions implemented by the Antimicrobial Management Team from Apr. 3, 2001 through Dec. 31, 2003, and the acceptance rates for each intervention type. During this period 1426 antimicrobial orders resulted in an intervention. Recommendations to change IV antibiotics to the PO route were accepted (i.e., not countermanded) 92% of the time. Recommendations to discontinue surgical prophylaxis at 24 hours were accepted 57% of the time. Recommendations to either discontinue restricted antibiotics or to obtain an infectious disease consult were accepted 87% of the time. In the majority of cases (54%), the drug was discontinued or changed to a non-restricted antibiotic by the prescribing physician; in a minority (46%) an infectious disease consultation was requested. The overall acceptance rate for all implemented interventions was 76%.

TABLE 2
ANTIMICROBIAL PROGRAM INTERVENTIONS
(APR. 3, 2001-DEC. 31, 2003)

| Intervention | IV to PO | Surgical | Restricted | |
|---|---|---|---|---|
| Type | Conversion | Prophylaxis | Antimicrobials | Total |
| Total No. of | 328 | 585 | 513 | 1426 |
| Interventions | | | | |
| No. Accepted | 301 (92%) | 336 (57%) | 447 (87%) | 1084 |
| | | | | (76%) |
| No. Rejected | 27 | 249 | 66 | 342 |

IV to PO Conversion

Compliance with IV to PO conversion has been nearly 100% since July 2001, when pharmacists began automatically converting appropriate patients from IV to oral antimicrobials. Prior to this, a note had been left asking the physician to make the change. The number of implemented interventions for IV to PO conversion has dropped dramatically, indicating that physicians are converting patients without intervention. To date, no evidence suggests that physicians have countermanded the changes from IV to PO made by the pharmacists.

Perioperative Prophylaxis

Compliance with the surgical prophylaxis has fluctuated. There has been a downward trend in the number of implemented interventions needed, with most physicians now ordering prophylactic antibiotics for 24 hours or less. When non-compliance with the surgical prophylaxis recommendations was analyzed by specialty, it was determined that most non-compliance was the result of three surgeons. Operative records and microbiology submitted from their cases were reviewed, and no evidence of infection was documented. This information was transmitted to their department chairman section chief and the Quality Resource Management Committee. Monitoring will continue and further information will be provided to the surgeons. Even though compliance with surgical prophylaxis recommendations has been lower than hoped, there has been a significant impact on the number of antibiotic doses used for surgical prophylaxis. Utilization of cefazolin, the antibiotic primarily used for surgical prophylaxis at the hospital of the study, decreased significantly from 2000 to 2003 (see Table 4a).

[image]

Restricted Antimicrobials

Compliance with implemented interventions on restricted antimicrobials has remained high and the number of interventions has remained fairly constant Tables 4b, 4c and 4d show a statistically significant decrease in the DDD per 1000 patient days for cefepime, ceftazidime and imipenem, respectively. Because of supply problems with meropenem, there was insufficient usage to draw any conclusion. Vancornycin and linezolid use increased despite restriction, most likely due to the increasing prevalence of community acquired MRSA (see Tables 4e and 4f).

[image]

[image]

[image]

[image]

Effect on Antibiotic Cost

Antimicrobial drug cost per census day decreased from $13.67 in 2000 (prior to program implementation) to $9.41 in 2003 (see Table 5). Total acquisition cost savings were $1,841,203 for the year period. This cost savings is based on antimicrobial aquisition cost alone and does not include mixing costs, administration costs, tubing cost, or nursing and pharmacy time.

[image]

Effects on Susceptibility

Comparisons between 2000, the last year before implementation of the study and 2002 and 2003, the first and second full years of the study, revealed no statistically significant change in susceptibility of Enterobucter cloucue (134 isolates), Proteus mirahilis (133), non-urinary Pseuclomonas aeruginosa (338) and Serratia marcescens (109) to ceftazidiine and cefepime. Susceptibility of non-urinary E. coli (548) to cefepime decreased significantly (100% to 98%), but change in susceptibility to ceftazidime was not significant. Susceptibility of urinary E. coli (1615) to ceftazidime fell significantly (100% to 98%). Despite decreased use, Pseudomonas aeruginosa susceptibility to imipenem decreased significantly from 93% in 2000 to 83% in 2002. Susceptibility improved in 2003 (91 %). Among 218 isolates of Klebsiella pneumoniae, there was a statistically significant increase in susceptibility to the following antimicrobials (see Table 3): ceftazidime, ceftriaxone, cefuroxime, levofloxacin, gentamicin, tobramycin and trimethoprirn/sulfa. Interestingly, only ceftazidime and cefepime were restricted by our program. The change in susceptibility to cefepime did not reach statistical significance (p>0.05).

TABLE 3
CHANGES IN K. PHEUMO SUSCEPTIBILITY

| | | Percent | Percent |
|---|---|---|---|
| | Percent Susceptible | Susceptible | Susceptible |
| ANTIMICROBIAL | in 2000 | in 2002 | in 2003 |
| Cefepime | 94 | 98 | 98 |
| Ceftazidime | 85 | 99* | 96* |
| Ceftriaxone | 85 | 98* | 96* |
| Cefuroxime | 78 | 94* | 93* |
| Gentamicin | 83 | 99* | 96 |
| Levofloxacin | 82 | 99* | 96* |
| Tobramycin | 90 | 99* | 96 |
| Trimethoprirn/Sulfa | 86 | 97* | 93 |

*Statistically significant change compared to 2000 (p = <0.05)

Discussion

While not being bound by theory, it is believed that our program has been successful for many reasons. Prior to the program, an entire year was devoted to educating physicians about the importance of the program and gaining their support. Support from the Hospital Administration has also contributed to the success of the program, and we were able to conduct the study with dedicated personnel from the Department of Pharmacy and the Department of Microbiology. Having an Infectious Disease physician as the program champion also contributed to the success. Conducting the program was labor intensive, especially during the planning and initial stages. Costs of the program included two pharmacists (1.0 FTE), a microbiologist (0.25 FTE) and a physician (30 hours per month). The antibiotic team met three to four times a week for the first nine months of the program. The program has become less labor intensive with time. The team now meets once weekly to review the results of surveillance of antibiotic usage. The antibiotic team works closely with the Infection Control and Microbiology Departments to monitor microbial resistance patterns and detect trends quickly. Overall antibiotic utilization is also monitored to detect new trends indicating inappropriate antibiotic use.

It should be recognized that most programs implemented in U.S. hospitals have been in response to an increase in multi-drug resistant organisms, and have made reduction in resistance one of the major goals. Our program differs in that we have not yet experienced an increase in multi-drug resistance, with the exception of the dramatic increase in prevalence of methicillin resistant StaphyIococcus aureus (MRSA). It is suspected that we were unable to reduce the use of vancomycin or linezolid because of the increase in community acquired MRSA. However, the requirement for an Infectious Disease physician consultation after 48 hours of use frequently allowed us to change therapy to drugs such as trimethoprim sulfa or minocycline. The improvement in susceptibility patterns of Klehsiella pneumoniae was unexpected (and gratifying).

In view of the above, it is believed that the primary benefit (but not the only benefit) of the CAMP intervention is a reduction in inappropriate antibiotic use without adverse patient outcomes. This reduction in inappropriate antibiotic use can slow or prevent the emergence of resistant organisms, which in turn leads to reduced morbidity, mortality and healthcare costs.

While the invention has been described in the specification and illustrated in the drawings with reference to a preferred embodiment, it will be understood by those skilled in the art that various changes may be made and equivalents may be substituted for elements thereof without departing from the scope of the invention as defined in the claims. In addition, many modifications may be made to adapt a particular situation or material to the teachings of the invention without departing from the essential scope thereof. Therefore, it is intended that the invention not be limited to the particular embodiment illustrated by the drawings and described in the specification as the best mode presently contemplated for carrying out this invention, but that the invention will include any embodiments falling within the foregoing description and the appended claims. The references cited herein are incorporated herein by reference in their entirety.

We claim:

1. A protocol for an integrated management of medical risk, said protocol comprising:
establishing at least one first intervention,
said first intervention in integration with at least one second intervention;
wherein said protocol provides said integrated management of medical risk.

2. The protocol of claim 1, comprising a plurality of interventions, wherein said plurality of interventions in integration provides for said integrated management of medical risk.

3. The protocol of claim 1, wherein said protocol is included as part of a software program or a system of risk management.

4. The protocol of claim 1, wherein said first or second intervention is an intervention for management of antimicrobial therapy.

5. The protocol of claim 4, wherein said intervention for management of antimicrobial therapy includes at least one component selected from the group consisting essentially of (a) converting a patient from intravenous administration to oral administration of at least one antimicrobial; (b) limiting patient prophylaxis to a pre-operative, post-operative, or combination thereof length of time; (c) restricting at least one antimicrobial from administration to said patient; and (d) combinations thereof.

6. The protocol of claim 4, wherein said intervention for management of antimicrobial therapy comprises converting a patient from intravenous administration to oral administration of at least one antimicrobial.

7. The protocol of claim 4, wherein said intervention for management of antimicrobial therapy comprises limiting patient prophylaxis to a pre-operative, post-operative, or combination thereof length of time.

8. The protocol of claim 4, wherein said intervention for management of antimicrobial therapy comprises restricting at least one antimicrobial from administration to a patient.

9. The protocol of claim 1, wherein said first or second intervention is an intervention for infection control, wherein said intervention for infection control is selected from the group consisting essentially of (a) standardizing definitions, polices and procedures; (b) targeted surveillance of surgical site infections; (c) targeted surveillance of intensive care unit infections; (d) rapid response to crises; (d) standardizing employee health procedures; and (e) combinations thereof.

10. The protocol of claim 1, wherein said first or second intervention is an intervention for control of surgical site infection.

11. The protocol of claim 10, wherein said intervention for control of surgical site infection is a non-pharmacologic intervention selected from the group consisting essentially of (a) skin preparation prior to surgery; (b) supplemental oxygen during surgery; (c) maintenance of body temperature during surgery; (d) maintenance of blood sugar during and after surgery; and (e) combinations thereof.

12. The protocol of claim 10, wherein said intervention for control of surgical site infection is a pharmacologic intervention selected from the group consisting essentially of (a) administration of perioperative antibiotics; (b) analysis of process procedures; (c) analysis of process outcomes; (d) modification of process based on the analysis of process procedures and outcomes; and (e) combinations thereof.

13. The protocol of claim 1, wherein said first or second intervention is an intervention for laboratory control, wherein said intervention for laboratory control includes at least one component selected from the group consisting of (a) standardizing laboratory procedures; (b) restricting cultures submitted to the laboratory; (c) restricting susceptibility reporting; and (e) combinations thereof.

14. A protocol for management of medical risk, said protocol comprising:
establishing a first intervention for management of antimicrobial therapy, said first intervention comprising:
(a) converting a patient from intravenous administration to oral administration of at least one antimicrobial;
(b) limiting patient prophylaxis to a pre-operative, post-operative, or combination thereof length of time;
(c) restricting at least one antimicrobial from administration to said patient; or
(d) a combination of components (a), (b) and (c).

15. The protocol of claim 14, wherein said first intervention is in integration with at least one second intervention.

16. The protocol of claim 14, further comprising at least one third intervention, wherein said third intervention is in integration with said first intervention, second intervention, or a combination of first and second interventions.

17. The protocol of claim 14, wherein said protocol is included as part of a software program or a system of risk management.

18. The protocol of claim 14, wherein said first or second intervention is an intervention for infection control, wherein said intervention for infection control is selected from the group consisting essentially of (a) standardizing definitions, polices and procedures; (b) targeted surveillance of surgical site infections; (c) targeted surveillance of intensive care unit infections; (d) rapid response to crises; (d) standardizing employee health procedures; and (e) combinations thereof.

19. The protocol of claim 14, wherein said first or second intervention is an intervention for control of surgical site infection.

20. The protocol of claim 19, wherein said intervention for control of surgical site infection is a non-pharmacologic intervention selected from the group consisting essentially of (a) skin preparation prior to surgery; (b) supplemental oxygen during surgery; (c) maintenance of body temperature during surgery; (d) maintenance of blood sugar during and after surgery; and (e) combinations thereof.

21. The protocol of claim 19, wherein said intervention for control of surgical site infection is a pharmacologic intervention selected from the group consisting essentially of (a) administration of perioperative antibiotics; (b) analysis of process procedures; (c) analysis of process outcomes; (d) modification of process based on the analysis of process procedures and outcomes; and (e) combinations thereof.

22. The protocol of claim 14, wherein said first or second intervention is an intervention for laboratory control, wherein said intervention for laboratory control includes at least one component selected from the group consisting of (a) standardizing laboratory procedures; (b) restricting cultures submitted to the laboratory; (c) restricting susceptibility reporting; and (e) combinations thereof.

23. A method for integrated management of medical risk, said method comprising:
establishing at least one first intervention,
said first intervention in integration with at least one second intervention;
wherein said method provides said integrated management of medical risk.

24. The method of claim 23, comprising a plurality of interventions, wherein said plurality of interventions in integration provides for said integrated management of medical risk.

25. The method of claim 23, wherein said protocol is included as part of a software program or a system of risk management.

26. The method of claim 23, wherein said first or second intervention is an intervention for management of antimicrobial therapy.

27. The method of claim 26, wherein said intervention for management of antimicrobial therapy includes at least one component selected from the group consisting essentially of (a) converting a patient from intravenous administration to oral administration of at least one antimicrobial; (b) limiting patient prophylaxis to a pre-operative, post-operative, or combination thereof length of time; (c) restricting at least one antimicrobial from administration to said patient; and (d) combinations thereof.

28. The method of claim 26, wherein said intervention for management of antimicrobial therapy comprises converting a patient from intravenous administration to oral administration of at least one antimicrobial.

29. The method of claim 26, wherein said intervention for management of antimicrobial therapy comprises limiting patient prophylaxis to a pre-operative, post-operative, or combination thereof length of time.

30. The method of claim 26, wherein said intervention for management of antimicrobial therapy comprises restricting at least one antimicrobial from administration to a patient.

31. The method of claim 23, wherein said first or second intervention is an intervention for infection control, wherein said intervention for infection control is selected from the group consisting essentially of (a) standardizing definitions, polices and procedures; (b) targeted surveillance of surgical site infections; (c) targeted surveillance of intensive care unit infections; (d) rapid response to crises; (d) standardizing employee health procedures; and (e) combinations thereof.

32. The method of claim 23, wherein said first or second intervention is an intervention for control of surgical site infection.

33. The method of claim 32, wherein said intervention for control of surgical site infection is a non-pharmacologic intervention selected from the group consisting essentially of (a) skin preparation prior to surgery; (b) supplemental oxygen during surgery; (c) maintenance of body temperature during surgery; (d) maintenance of blood sugar during and after surgery; and (e) combinations thereof.

34. The method of claim 32, wherein said intervention for control of surgical site infection is a pharmacologic intervention selected from the group consisting essentially of (a) administration of perioperative antibiotics; (b) analysis of process procedures; (c) analysis of process outcomes; (d) modification of process based on the analysis of process procedures and outcomes; and (e) combinations thereof.

35. The method of claim 23, wherein said first or second intervention is an intervention for laboratory control, wherein said intervention for laboratory control includes at least one component selected from the group consisting of (a) standardizing laboratory procedures; (b) restricting cultures submitted to the laboratory; (c) restricting susceptibility reporting; and (e) combinations thereof.

36. A method for management of medical risk, said method comprising:
establishing a first intervention for management of antimicrobial therapy, said first intervention comprising:
(a) converting a patient from intravenous administration to oral administration of at least one antimicrobial;
(b) limiting patient prophylaxis to a pre-operative, post-operative, or combination thereof length of time;
(c) restricting at least one antimicrobial from administration to said patient; or
(d) a combination of components (a), (b) and (c);
wherein said method provides said management of medical risk.

37. The method of claim 36, wherein said first intervention comprises components (a), (b) and (c).

38. The method of claim 36, wherein said first intervention is in integration with at least one second intervention.

39. The method of claim 36, wherein said method is included as part of a software program or a system of risk management.
